(19) United States
(12) Patent Application Publication
(10) Pub. No.: US 2003/0026852 A1
(43) Pub. Date: Feb. 6, 2003

(54) Molecular iodine pharmaceutical composition

(75) Inventors: Yongjun Duan, Lexington, MA (US); Jack Kessler, Southborough, MA (US)

Correspondence Address: Anderson, Kill & Olick, P.C., 1251 Avenue of the Americas, New York, NY, 10020-1182, US

(73) Assignee: SYMBOLLON CORPORATION

(21) Appl. No.: 09/818,477

(22) Filed: Mar. 27, 2001

(51) Int. Cl. A61K 33/18; A61K 47/08

(52) U.S. Cl. 424/667; 514/784

(57) ABSTRACT

This invention describes a non-aqueous oral composition of molecular iodine in a stabilized hydrophobic environment containing a hydrophobe or combination of hydrophobes for treating different disease states.

### FIELD OF THE INVENTION

This invention relates to pharmaceutical compositions of molecular iodine stabilized in a hydrophobic environment for oral administration to mammals and more particularly to a stable oral composition containing molecular iodine and other iodine species with the ratio of molecular iodine to such other iodine species controlled such that the concentration of the iodine-containing species other than molecular iodine is minimized to 10% or less of the concentration of total iodine in the composition.

### BACKGROUND

Iodine is an essential element in human nutrition. Iodine deficiency is responsible for a host of diseases and remains the leading cause of mental retardation in the world. Iodine has been reported to have a number of potential therapeutic applications. Thrall and Eskin (Thrall K D. Bull R J. Fund. App. Tox. 1990;15:75-81; Thrall K D. Formation of Organic By-Products Following Consumption of Iodine Disinfected Drinking Water. Ph.D. Dissertation, December 1990.; Eskin B A, Grotowski C E, Connolly C P, Ghent W R. Biological Trace Element Research, 1995;49: 9-18) have demonstrated that molecular iodine is less toxic than iodide when administered orally. Currently, there are no commercially available compositions of pure molecular iodine which are suitable for oral administration to mammals. The only form of pure molecular iodine that is commercially available is metallic elemental iodine which are suitable for oral administration to mannals. The only form of pure molecular iodine that is commercially available is metallic elemental iodine. A number of practitioners have developed approaches for the oral administration of iodine. Most of these approaches suffer by virtue of the fact that molecular iodine sublimes at room temperature and is unstable in the presence of water.

The most serious concern for administration of an iodine pharmaceutical relates to compositions relates to the potential for iodide poisoning, or “iodism.” There is no way of predicting which patient will react unfavorably to iodide, and an individual may vary in their sensitivity to iodide from time to time. A series of symptoms can result from iodism. Symptoms can include burning in the mouth and throat; soreness of the teeth and gum; increased salivation; coryza, irritation of the respiratory tract; cough; headache; enlarged glands; inflammation of the pharynx, larynx and tonsils; skin lesions; gastric irritation; diarrhea; fever; anorexia and depression; and severe and sometimes fatal eruptions (ioderma) may occur. In essence, human consumption of iodide at levels above the upper limit established by the U.S. Federal Drug Agency, 150 to 1,000 μg/day, presents a health risk (J. A. Pennington, “A review of iodine toxicity reports”, J. Am. Dietetic Assoc., 1990; Vol. 90, pp. 1571-1581.

It is known that iodide can negatively effect mammalian thyroid parameters at concentrations that are 10 fold less than a comparable effect from molecular iodine (Sherer T T, Thrall K D, Bull R J. J. Tox. Env. Health 32, 89-101 (1991)). Another way to state this is that, up to now, ten times or more molecular iodine is required relative to iodine to effect animal thyroid function with an orally administered iodine composition. The relationship between the desired and undesired effects of a drug is termed its therapeutic index or selectivity. In clinical studies, drug selectivity is often expressed indirectly by summarizing the pattern and nature of adverse effects produced by therapeutic doses of the drug and by indicating the proportion of patients with adverse side effects. Each separate iodine species should be considered to be a unique drug entity since each has been shown to have a different oral toxicity from the others. As compared to iodide, molecular iodine's toxicological profile in mammals makes it the preferred form of iodine to treat iodine deficiency diseases. Therefore, a preferred “iodine” therapeutic is a composition wherein all or an overwhelming majority of the total iodine present is in the desired form.

Given the favorable toxicity profile of molecular iodine as compared to iodide it is not surprising that a large number of practitioners have attempted to develop an acceptable pharmaceutical dosage form of molecular iodine. Efforts in this regard have been stymied by the fundamental properties of molecular iodine. Elemental iodine is a soft metal that sublimes at room temperature and interacts with water to yield a large number of diverse iodine species. These unfavorable properties have made it extraordinarily difficult to develop a stable oral dosage form of molecular iodine.

U.S. Pat. No. 4,816,255 claims the use of a pure aqueous solution of molecular iodine to treat iodine deficiency diseases. Pure aqueous molecular iodine is claimed to have advantages as compared to Lugol's solution (improved taste) and iodine crystals (adverse reactions) as described in U.S. Pat. Nos. 4,384,960. 5,171,582, 5,250,304, and 5,389,385 all contain teachings that are substantially identical to U.S. Pat. No. 4,816,255 and teach the oral administration of a pure aqueous solution of molecular iodine. However, aqueous molecular iodine is not stable. In fact, depending upon the pH of the environment, molecular iodine can exhibit a half-life that is extremely short i.e., seconds.

U.S. Pat. No. 5,589,198 claims the oral administration of elemental iodine in combination with a suitable pharmaceutical excipient and the same inventors further describe the embodiment of U.S. Pat. No. 5,589,198 in WO 92/17190. WO 92/17190 describes dry powder formulations that use starch to complex elemental iodine. Such compositions are used to prepare dry pharmaceutical formulations used to make capsules or tablets. Such capsules provide a solid composition for oral administration of molecular iodine. However, these solids still experience the problems associated with hydration from trace amounts of water and would be anticipated to suffer from sublimation.

One approach to overcome these problems is taught in Duan et. al (U.S. Pat. No. 5,885,592) which does not include molecular iodine in the formulation of the oral composition but instead generates this species from precursors in situ after administration.

DEFINITION OF TERMS

For convenience, certain terms employed in the specification, examples, and appended claims are defined below.

The term “molecular iodine” as used herein, refers to diatomic iodine.

The term “elemental iodine” as used herein, refers to diatomic iodine in the solid state, which is represented by the chemical symbol I₂.

The term “iodide” or “iodide anion” refers to the species which is represented by the chemical symbol I⁻. Suitable cations for the iodide anion include sodium, potassium, calcium, and the like.

The term “triiodide” refers to the species which is represented by the chemical symbol I₃⁻. It is recognized by one skilled in the art that triiodide is formed from the association of one iodide anion and one molecule of molecular iodine and that triiodide rapidly dissociates into one iodide anion and one molecule of molecular iodine.

The term “total iodine” as used herein, refers to the following iodine species: elemental iodine, molecular iodine, iodide, organically complexed forms of iodine, covalently bound forms of iodine, iodite, triiodide and other polyiodides.

The term “ratio of molecular iodine” as used herein, refers to the ratio of molecular iodine (I₂) to total iodine. This ratio has a range between 0 and 1.0 where a ratio of 1.0 indicates a composition of matter that only contains molecular iodine without contamination from any other iodine species.

The term “hydrophobe” as used herein, refers to an organic molecule which is substantially water insoluble which provides a hydrophobic environment such that molecular iodine is stabilized. A hydrophobe can consist of mixtures of organic molecules with differing iodine stabilizing properties.

### PRIOR ART

Ghent (U.S. Pat. No. 5,389,385) has performed research using, what he believed to be, “pure” aqueous solutions of molecular iodine. However, pure aqueous solutions of molecular iodine do not exist in commerce. Molecular iodine is known to be unstable in water and is lost via several mechanisms. Molecular iodine is hydrated by water and, in an aqueous system, immediately undergoes the series of reactions shown below in equations 1 to 3.

I₂+H₂O=HOI+I⁻+H⁺ (1)

3HOI=IO₃³¹+2I⁻ (2)

I₂+I⁻=I₃⁻ (3)

The prior art demonstrates that molecular iodine is at least as effective as iodide when considered as a therapeutic agent in a number of iodine deficiency disease states. The scientific literature also indicates that the oral toxicity of iodide is materially greater than that for molecular iodine. Another way to state this is to say that the prior art demonstrates that the most therapeutic form of iodine when administered orally is molecular iodine due to its lower toxicity. Therefore, the prior art indicates that all of the iodine in a preferred oral iodine pharmaceutical should be molecular iodine.

Since the toxicity of an oral pharmaceutical iodine drug is directly related to the ratio and concentration of the different iodine species present, the known instability of the I₂ species presents a challenge to the development of an oral iodine pharmaceutical composition with a preferred therapeutic index.

### SUMMARY OF THE INVENTION

The present invention describes non-aqueous compositions of molecular iodine in a hydrophobic environment which are stable and suitable for oral administration to mammals to treat iodine deficiency diseases. These compositions include molecular iodine and a compound forming a hydrophobic environment for the molecular iodine which will eliminate intimate contact between molecular iodine and water. As compared to aqueous compositions of pure molecular iodine the compositions identified herein overcome stability problems since water is not in contact with molecular iodine. This overcomes the problems of the prior art in the delivery of molecular iodine in combination with a suitable pharmaceutical excipient for forming a pharmaceutically acceptable oral composition.

In one aspect of the present invention, molecular iodine is dissolved in a hydrophobic carrier such as, for example, mineral oil, petrolatum and paraffin and can be further dispersed in or combined with additional hydrophobic carriers. If additional iodine species are included, an accurate dose of molecular iodine having a desired ratio of molecular iodine to total iodine is administrated in the composition with the hydrophobe to a human and/or other mammals in the treatment of a given disease state. Molecular iodine has been shown to exert a beneficial effect for a number of different disease states. Some of these disease states are short lived and others are chronic conditions.

The pharmaceutical compositions of the present invention comprise molecular iodine in a non-aqueous environment provided by a hydrophobe or a combination of hydrophobes. The role of the a non-aqueous environment is to stabilize molecular iodine. Any hydrophobic material which will maintain the molecular iodine in a non-aqueous state will be suitable including hydrophobic materials in liquefied form such as oils that are hydrophobic molecules and/or emulsions containing said hydrophobic molecules. It is also feasible to utilize hydrophobes that form gels and waxes at certain temperatures alone or in combination with other hydrophobes.

Some of the advantages of administering molecular iodine in a hydrophobic environment are: (1) it is easy and inexpensive to produce a stable hydrophobic composition; (2) an accurate amount of molecular iodine can be provided in each dose; (3) it is possible to deliver iodine such that the ratio of molecular iodine to total iodine is minimally 0.90 and can equal 1.0; and (4) the stability of the molecular iodine is increased in a hydrophobic environment. Moreover, experiments described in this application demonstrate that the toxicity of molecular iodine is reduced when it is introduced in combination with a hydrophobe as compared to an aqueous environment, although it is not yet understood why such disparity exists.

The present invention allows an accurate dosage regime to be achieved to reduce the unwanted side effects that are associated with iodide or other aqueous reaction products from iodine hydrolysis such as trioxide.

Broadly, the present invention relates to a non-aqueous oral iodine pharmaceutical composition comprising molecular iodine and a hydrophobe having the general formula

H₃—X—(CH₂)ₙ—Y

wherein X is selected from the group consisting of —CH₂—, ═CHOH, ═CHCOOH, ═CO, ═CHCHO, ═CHCOOR, C(HO—CH₂R)—, —O—, and —NH₂; and Y is selected from the group consisting of —CH₃, —CH₂OH, —CH₂COOH, —CH₂O, —C(H₂COOR)—, —CH₂O—CH₂R, , and —NH₂; n is an integer between 8 and 26; where p is an integer between 1 and 5; R is selected from the group consisting of —H, —(CH₂)ₚ—CH₃.

### DETAILED DESCRIPTION OF THE INVENTION

The present invention describes a stabilized molecular iodine oral pharmaceutical composition that has an advantageous therapeutic index to different disease states. There are three key technical elements to the stability of molecular iodine in anpharmaceutical composition. The first technical element is to provide a proper matrix which will contain an acceptable level of iodine reactive chemical moieties (e.g., double bonds, sulfhydryl groups). The second technical element is to provide an environment that (a) does not contain water or (b) allow water or moisture to gradually come into contact with molecular iodine. The third technical element is to provide an environment in which molecular iodine is thermodynamically stable.

### DETAILED DESCRIPTION OF THE INVENTION

Molecular iodine is known to react with a wide range of chemical functionalities including alkenes, alkynes, primary alcohols, diazonium cations, amines and sulfhydryl groups. In order to provide a stable pharmaceutical composition it is important to consider the reactivity of molecular iodine and it is necessary to select excipients and carriers for the matrix that do not contain such iodine-sensitive functional groups. Alternatively, it is possible to control conditions and concentrations such that an acceptable degree of interaction occurs between said iodine-sensitive function groups and molecular iodine. This can be accomplished by combining various hydrophobic carriers that have a different reactivity with molecular iodine. The latter approach can result in hydrophobic carrier compositions that contain different molecules with various functional groups such as alcohols, ethers, unsaturated and saturated hydrocarbons. For instance, it may be acceptable to combine mineral oil that contains molecular iodine with oleic acid such that the oleic acid is 5% by weight of the final composition. Even though oleic acid contains a double bond that will react with molecular iodine, the change in the ratio of molecular iodine to total iodine may be within a range that is acceptable depending upon the initial concentration of molecular iodine.

Molecular iodine is a hydrophobic molecule that is easily polarized. The reaction of molecular iodine with water that leads to loss of molecular iodine is well known and has been discussed in this application. Because of molecular iodine's hydrophobic nature it is possible to incorporate molecular iodine into a hydrophobic environment. For instance, mineral oil provides a suitable hydrophobic environment. Mineral oil does not react with iodine since it is a mixture of saturated hydrocarbons. If molecular iodine is dissolved in a non-aqueous hydrophobic environment, such as that provided by mineral oil, the stability of molecular iodine is dramatically increased relative to its stability in an environment that contains water or an environment that sequesters water from the atmosphere. Examples of this increased stability are provided in the Example Section, of this application.

For the purposes of this invention, a wide range of materials are capable of acting as a hydrophobe to stabilize molecular iodine. The three broad classes of compounds that are acceptable are (1) non-aqueous liquids such as oils, (2) hydrophobic semisolids or gels, and (3) hydrophobic solids or waxes. Examples of the first class include mineral oil and silicone oil. An example of the second class is petrolatum. Examples of the third class are waxes like hexacosane or hexacontane.

Oils, gels and waxes that are suitable to act as a hydrophobe as defined in this application can be prepared synthetically or found in nature. Hydrophobes are organic molecules that are, as a general rule, water insoluble. The structural backbone for these molecules is a linear sequence of carbon atoms as represented by the class of organic molecules known as alkanes. Other functional groups that fall within the scope of a hydrophobe for the purposes of this application include branched alkanes, cyclic alkanes, ketones, acids, esters, secondary alcohols, polyesters, diesters, terpenoids, phospholipids and triglycerides. Examples of hydrophobes include: alkanes like mineral oil and petrolatum; saturated fatty acids like lauric acid, myristic acid, palmitic acid, stearic acid, arachidic acid and lignoceric acid; and secondary alcohols like cerebronic acid and dodecanoic alcohol. A suitable hydrophobe may be selected from the group consisting of cetostearyl alcohol, polyoxyethylene derivatives of sorbitan, straight chain hydrocarbons, branched-chain hydrocarbons, cyclic hydrocarbons, white wax, yellow wax, hydrogenated vegetable oil, carnuba wax, triglycerides of stearic acid, triglycerides of palmitic acid, tocopherols, squalene, soybean oil, sorbitan monolaurate, sorbitan monoleate, sorbitan monopalmitate, sodium stearate, sodium palmitate, sesame oil, rose oil, sorbitol derivatives commonly called polysorbates, monostearate derivatives commonly called polyoxy stearates, castor oil, polyoxyl ethylene diol derivitives such as polyoxyl 10 oleyl ether or polyoxyl 20 cetostearyl ether, polyethylene oxide, polyethylene glycol, peppermint oil, paraffin, olive oil, oleyl alcohol, oleic acid, octydodecanol, octoxynol 9, nonoxynol 10, myristyl alcohol, light mineral oil, lanolin alcohol, lanolin, isopropyl palmitate, isopropyl myristate, glycerol monostearate, glycerol behenate, oleate, cottonseed oil, cetyl alcohol, cetostearyl alcohol, cetyl esters wax, hydrogenated castor oil, butyl paraben, almond oil, soybean oil, simethicone, safflower oil, panthenol, mineral oil, common unsaturated fats include, oleic, linoleic, linolenic and arachidonic acids. Edible fats and oils are mixtures that contain saturated and unsaturated fatty acids. For instance, soybean oil contains myristic, palmitic, stearic, oleic, linoleic, linolenic, arachidic, and eicosenoic acids. Other commonly consumed oils include cottonseed oil, canola oil, olive oil, corn oil, peanut oil, safflower oil, palm oil and sunflower oil.

For the purposes of this application alkanes with at least 6 carbon atoms and preferably at least 8 carbon atoms are acceptable. Representative examples of these alkanes include octane, docosane, tetracosane and eicosane.

For the purposes of this application, for each nine carbon atoms in a hydrophobe, no more than one of the following seven functional groups should be present: alcohols (—OH), acids (—COOH), aldehydes (—CHO), ketones (═CO), esters (—COOR), ethers (—COC—)or amines (═NH). Suitable alkenes and alkynes derived from the alkanes identified above may be used provided that the concentration of said unsaturated hydrocarbons does not decrease the ratio of moleuclar iodine to total iodine to a value that is less than 0.9.

Unsaturated fats are generally not preferred since their double bonds react with iodine but small amounts of unsaturated fats can be used to provide certain features. For instance, an unsaturated fat may provide lubricity in a manufacturing process and improve the bioavailability of the composition. Alternatively, unsaturated fats can be hydrogenated in order to reduce their ability to react with molecular iodine. Common unsaturated fats include, oleic, linoleic, linolenic and arachidonic acids. Edible fats and oils are mixtures that contain saturated and unsaturated fatty acids. For instance, soybean oil contains myristic, palmitic, stearic, oleic, linoleic, linolenic, arachidic, and eicosenoic acids. Other commonly consumed oils include cottonseed oil, canola oil, olive oil, corn oil, peanut oil, safflower oil, palm oil and sunflower oil.

Treatment of breast dysplasia is an example of a disease that requires chronic dosing. The amount of molecular iodine delivered per day for chronic dosing can be between 1.0 and 15 mg with a preferred range of iodine for consumption between 1.5 and 8.0 mg per day.

An important parameter of any iodine pharmaceutical is its therapeutic index. The therapeutic index for an iodine pharmaceutical is proportional to the ratio of molecular iodine to total iodine contained in said pharmaceutical. The higher the ratio of molecular iodine to total iodine, the higher the therapeutic index for the iodine composition. The ratio of molecular iodine to total iodine for iodine pharmaceuticals described in this application must be between 0.9 and 1.0 with a preferred ratio of between 0.95 and 1.0. In order to limit toxicity from unwanted iodide species to no more than the toxic effect due to molecular iodine, it is necessary to limit the concentration of iodide by weight to no more than 10% of the weight of the total iodine present while maintaining a ratio (based on concentration) of at least 0.9 of molecular iodine.

The stability of the composition contemplated in this application should be such that the minimal 0.9 ratio of molecular iodine that is initially present remains constant after storage in appropriate packaging for at least 3 months and preferably 6 months. It is very important that the ratio of molecular iodine to total iodine does not materially change during storage.

### EXAMPLES

### Example 1

The intent of this experiment was to determine if there is a difference in the oral toxicity of aqueous iodine as compared to molecular iodine in a hydrophobe. Eight week old mice (C57BL/6 strain) were obtained and housed in a micro-isolator cage under germ-free conditions. Mice were allowed unrestricted access to food and water. Mice were divided into four groups of five animals and each animal was dosed with iodine compositions twice daily for two weeks. A control group of five animals was also maintained throughout the course of these studies. The volume used for intragastric instillation was 0.5 mL.

Aqueous iodine compositions with defined concentrations of molecular iodine were formed by reacting horseradish peroxidase with sodium iodide and hydrogen peroxide. A buffer solution was prepared for these experiments that contained 50 grams of anhydrous citric acid and 125 grams of sodium citrate for every 500 ml of buffer. Five milligrams of horseradish peroxidase was added into 500 mL of buffer and the following three different reaction conditions were established: (1) hydrogen peroxide 0.06%, sodium iodide 1.5 grams per liter; (2) hydrogen peroxide 0.06%, sodium iodide 3.0 grams per liter; and (3) hydrogen peroxide 0.12%, sodium iodide 6.0 grams per liter. The final pH of these reactions was 5.1. The concentration of molecular iodine for these different reaction conditions was: (1) 260 ppm; (2) 235 ppm and (3) 151 ppm. The triiodide concentration for each of these reactions was (1) 429 ppm; (2) 907 ppm and (3) 2,006 ppm.

Molecular iodine was stabilized in mineral oil by establishing the reaction conditions described above and adding 200 mL of mineral oil on top of 500 mL of solution resulting from each reaction. The molecular iodine was extracted into the mineral oil by vigorously mixing the two phases and allowing them to settle. One mL of mineral oil containing the extracted iodine was mixed with nine mL of heptane and the absorbance was read in the UV at 521 nm. Stock solutions of known concentrations of molecular iodine yielded an absorbance in heptane of 0.142 at a concentration of 40 ppm in heptane. The concentration of molecular iodine in the mineral oil was: (1) 1,350 ppm; (2) 1,960 ppm, and (3) 3,160 ppm.

Mice that were dosed with the aqueous iodine solutions under conditions 1 and 2 exhibited diarrhea, they did not gain weight over the two week test period and they did not appear healthy; in addition, all of the mice in group 3 died within the first two days of the test period. At the end of the study the surviving mice were put to death and an autopsy was performed to examine their gastrointestinal (GI) tract; the GI tract of these animals did not appear normal.

Mice that were dosed with the non-aqueous iodine compositions all survived. All of the animals receiving the mineral oil-iodine composition gained weight, were healthy and exhibited normal behavioral patterns. No abnormalities in the GI tract was observed for these mice upon autopsy. It is not understood why these high concentrations of iodine in mineral oil were not toxic to the mice while lower concentrations of aqueous iodine were clearly toxic.

### Example 2

The stability of a solution of molecular iodine in oil was measured. Crystals of elemental iodine were dissolved at room temperature in mineral oil. The concentrations of molecular iodine were approximately 0.6, 3.0 and 6.0 mg/mL. The absorbance was determined at 522 nm as a measure of the concentration of molecular iodine. Samples were stored in 500 mL amber glass bottles (Fisher Cat. No. 05-719-466) with Teflon lined screw-tops. The screw tops contained a septum to sample the fluids using a syringe without opening the bottle. The bottles were stored in an incubator at 40° C.

Samples were analyzed monthly to determine if there was a loss of molecular iodine. On the day of an analysis, a bottle was removed from the incubator, placed into room-temperature water covering approximately ¾ of the bottle, and allowed to cool for at least 6 hours. A sample was removed with a glass syringe and its absorbance was determined at 522 nm. The results of these measurements are shown in Table 1 below: The data indicate that molecular iodine is stable in a hydrophobe at 40° C.

TABLE 1

| | Initial | Percentage of Initial Absorbance | | | | |
|---|---|---|---|---|---|---|
| mg/mL I₂ | OD₅₂₂ₙₘ | Day 14 | Day 32 | Day 56 | Day 97 | Day 143 |
| 0.6 | 0.325 | 99.5 | 99.1 | 98.2 | 97.8 | 96.4 |
| 3.0 | 1.674 | 100.6 | 100.5 | 100.4 | 100.1 | 100.8 |
| 6.0 | 3.167 | 100.3 | 100.2 | 99.2 | 101.4 | 100.7 |

Where OD referes to the optical density at the indicated wavelength.

### Example 3

The stability of molecular iodine in various hydrophobes and also an aqueous buffer were compared. Crystals of elemental iodine were dissolved at room temperature. The concentrations of molecular iodine used was approximately 3.0 mg/mL. For the oil based samples the absorbance was monitored and used as a measure of the concentration of molecular iodine. For the two aqueous samples the concentration of molecular iodine was measured according to a published potentiometric method (W. Gottardi,1983, Fresenius Z. Anal. Chem. 314:582-585). Samples were stored in 500 mL amber glass bottles (Fisher Cat. No. 05-719-466) with Teflon-lined plastic screw-tops. The bottles were stored in an incubator at 40° C.

Samples were incubated at 40° C. for 24 hours prior to analysis to allow them to reach equilibrium. Samples were then analyzed periodically to determine if there was a loss of molecular iodine. On the day of an analysis, a bottle was removed from the incubator, placed into room-temperature water covering approximately ¾ of the bottle, and allowed to cool for at least 4 hours. A potentiometric analysis for molecular iodine was performed directly in the storage bottle for the two aqueous samples. Evaporation of molecular iodine was eliminated by using a screw-top holder with holes for the required electrodes. For the oil-based samples an aliquot was removed from each bottle and their absorbance was determined at 522 nm. The percentage of the day 1 molecular iodine concentrations versus time for each sample is shown in Table 2 below: The data indicates that molecular iodine is substantially more stable in a hydrophobe than in an aqueous environment.

TABLE 2

| | Percent of Day 1 Molecular Iodine | | | | | |
|---|---|---|---|---|---|---|
| | Day 7 | Day 14 | Day 31 | Day 56 | Day 73 | Day 92 |
| 0.02 M | 24.5 | 8.8 | — | — | — | — |
| phosphate | | | | | | |
| pH 7.0 | | | | | | |
| 0.02 M | 88.9 | 73.2 | 57.8 | 23.6 | 12.1 | — |
| phosphate | | | | | | |
| pH 5.0 | | | | | | |
| light mineral | 92.9 | 85.85 | 68.66 | 43.39 | 26.21 | 93.03 |
| oil | | | | | | |
| heavy mineral | 92.42 | 92.42 | 92.42 | 92.42 | 92.42 | 99.6 |
| oil | | | | | | |
| mineral oil | 92.36 | 92.36 | 92.36 | 92.36 | 92.36 | 100.4 |
| Safflower oil | 94.45 | 94.45 | 94.45 | 94.45 | 94.45 | 73.65 |
| peanut oil | 93.76 | 93.76 | 93.76 | 93.76 | 93.76 | 82.19 |
| Squalene | 93.08 | 93.08 | 93.08 | 93.08 | 93.08 | 91.05 |
| Sorbitan | 94.22 | 94.22 | 94.22 | 94.22 | 94.22 | 76.43 |
| monolaurate | | | | | | |
| cetyl alcohol | 93.61 | 93.61 | 93.61 | 93.61 | 93.61 | 84.42 |

### Example 4

This experiment was designed to demonstrate the bio availability of molecular iodine that is carried in a hydro phobe. Female Sprague-Dawley rats weighing 150-250 grams that were 6-7 weeks old were purchased from Charles River Canada, Inc. (Quebec, Canada). Rats were housed individually in stainless steel wire mesh-bottomed rodent cages equipped with an automatic watering system. Following randomization, all cages were clearly labeled with a color-coded cage card indicating study number, group, animal number, sex and treatment. Each animal was uniquely identified by an individual ear tag following arrival. The environment was controlled at 21±3° C., 50±20% relative humidity, 12 hours light, 12 hours dark and 10-15 air changes made per hour. Animals were provided with Teklad (Madison, Wis.) Certified Rodent Diet (W) #8728 ad libitum. Municipal tap water that was purified by reverse osmosis and treated with ultraviolet light was provided ad libitum. The animals were allowed to acclimate to their environment for at least two weeks prior to the start of the experiment.

Rats were dosed with 1.0 ml per 250 grams for each treatment group. The concentration of molecular iodine was either 0.1 mg/kg (the low dose “L”) or 1.0 mg/kg (the high dose “H”). Molecular iodine in an aqueous environment i.e., Lugol's solution, was used as the positive control. Squalene was used as the hydro trope to carry molecular iodine.

Blood was drawn from animals prior to treatment. Animals were gavaged and blood was taken 24 hours later when the animals were sacrificed. The blood was processed to yield serum samples and these samples were frozen. The frozen samples were analyzed by utilizing the reduction-oxidation reaction between ceric and arsenite catalyzed by iodide. This method provides a measure of the total iodine that is absorbed in serum. The results of the these measurements are shown below in tabular form in Table 3.

TABLE 3

| Treatment Group | Conc. (mg/kg) | pre-dosing | 2 hr. post-dosing |
|---|---|---|---|
| Lugol's | H | 9.64 | 89.6 |
| | L | 9.37 | 11.3 |
| Squalene | H | 9.08 | 76.3 |
| | L | 9.38 | 15.7 |

The data indicate that molecular iodine is bioavailable to a mammal when provided in a hydrophobe.

### Example 5

The interaction of molecular iodine with mineral oil was evaluated. The following stock solutions were prepared for this experiment: 0.050 grams and 1.001 grams of elemental iodine crystals dissolved into about 80 ml of mineral oil in two different 100-ml flasks, and then filled to 100 ml with mineral oil. The absorbance spectrum between 300 and 600 nm for various mixtures of these two stock solutions were made in a UV/V is scanning spectrophotometer (Schimadzu 1125). The absorbance spectrums were analyzed and the extinction coefficient was calculated at appropriate wavelengths.

The UV-visible spectra of iodine-in-mineral oil presents two peaks in the range of 300-600 nm; one peak is at 302 nm and the other is at 522 nm (the maximum absorbance is at 522 nm). The absorbance is proportional to concentration as predicted by Beer's Law. The absorbance did not change with time and therefore the molecular iodine was stable in mineral oil. The two peaks are at 302 nm and 522 nm and are slightly variable in the test concentration range. The extinction coefficients (moles per 1000 grams)⁻¹ (per cm)⁻¹) at 302 and 522 nm were calculated to be 219 and 905 respectively.

### Example 6

The interaction of molecular iodine with a number of different oils was evaluated. A stock solution of iodine in mineral oil at a concentration of 0.6 mg/mL was prepared. This stock solution of mineral oil (0.50 mL) was mixed with 3.5 mL of the following edible oils: canola oil, vegetable oil, safflower oil, peanuts oil, corn oil, olive oil and mineral oil. After the oils were mixed the absorbance of the resulting solutions were recorded at 522 nm for 20 minutes. The initial absorbance and the absorbance after 20 minutes at room temperature at 522 nm for the different mixtures of these oils is shown below in Table 4.

TABLE 4

| Oil | Initial | 20 min. |
|---|---|---|
| Mineral oil | 0.308 | 0.309 |
| Canola oil | 0.181 | 0.150 |
| Vegetable oil | 0.162 | 0.121 |
| Safflower oil | 0.174 | 0.150 |
| Peanut oil | 0.157 | 0.137 |
| Corn oil | 0.129 | 0.106 |
| Olive oil | 0.132 | 0.104 |

All six oils reduced iodine absorbances in the range of 40-60% initially in comparison to mineral oil and additional 13-25% twenty minutes after initially forming the mixture. It is obvious from the data that all six of the edible oils tested react with iodine. Such a reaction leads to a reduction in the ratio of molecular iodine to total iodine.

### Example 7

Ghent and Eskin (Ghent W. R. and Eskin B. A., Can J Surg, 1993;36:453-60) dosed over 1,300 women daily for 6 months to 5 years with 3 to 6 mg per day of sundry forms of iodine all of which contained iodide to varying degrees. They published the adverse events that they observed. A number side effects were observed as indicated in the Table 5 below. As indicated above, the principal concern when dosing an iodine composition is the potential for toxicity to thyroid. Therefore, the incidence of hypothyroidism and hyperthyroidism observed in this study is of concern. In addition, the 2 cases of iodism are of note.

To determine the potential for molecular iodine to cause hyperthyroidism or hypothyroidism 111 women daily were dosed with either 0, 1.5, 3.0 or 6.0 mg of molecular iodine per day for six months. This study was performed under the guidance of the FDA (IND #56,523). The dosage form used relied upon in situ generation of molecular iodine as described in U.S. Pat. No. 5,885,592 as opposed to the aqueous iodine therapy of Table 5. The total exposure to molecular iodine in this study was 3.619 subject years. No hyperthyroidism or hypothyroidism was observed. In fact, the dose of molecular iodine was not associated with increases in incidence, severity and causality of treatment-emergent adverse events or clinically significant changes in laboratory parameters or vital signs. These results are consistent with prior published studies in rats.

TABLE 5
Side Effects of Aqueous Molecular Iodine Therapy in Women

| Side Effect | Number (%) of Women |
|---|---|
| Increased Breast Pain | 78(5.7) |
| Acne | 15(1.1) |
| Hair Thinning | 13(1.0) |
| Nausea | 8(0.6) |
| Hypothyroidism | 4(0.3) |
| Skin Rash | 3(0.2) |
| Headaches | 3(0.2) |
| Iodism | 2(0.1) |
| Hyperthyroidism | 2(0.1) |
| Diarrhea | 2(0.1) |
| Other | 22(1.6) |
| Total | 149(10.9) |

We claim:

1. A non-aqueous oral iodine pharmaceutical composition comprising molecular iodine and a hydrophobe, said hydrophobe having the general formula
CH₃—X—(CH₂)ₙ—Y
wherein X is selected from the group consisting of —CH₂—, ═CHOH, ═CHCOOH, ═CO, ═CHCHO, ═CHCOOR, C(HO—CH₂R)—, —O—, and —NH₂; and Y is selected from the group consisting of —CH₃, —CH₂OH, —CH₂COOH, —CH₂O, —C(H₂COOR)—, —CH₂O—CH₂R, , and —NH₂; n is an integer between 8 and 26; where p is an integer between 1 and 5; R is selected from the group consisting of —H, —(CH₂)ₚ—CH₃.

2. A non-aqueous oral iodine composition as defined in claim 1, wherein said hydrophobe is selected from the group consisting of cetostearyl alcohol, polyoxyethylene derivitives of sorbitan, straight chain hydrocarbons, branched-chain hydrocarbons, cyclic hydrocarbons, white wax, yellow wax, hydrogenated vegetable oil, carnuba wax, triglycerides of stearic acid, triglycerides of palmitic acid, tocopherols, squalene, soybean oil, sorbitan monolaurate, sorbitan monoleate, sorbitan monopalmitate, sodium stearate, sodium palmitate, sesame oil, rose oil, sorbitol derivatives commonly called polysorbates, monostearate derivitives commonly called polyoxy stearates, castor oil, polyoxyl ethylene diol derivitives such as polyoxyl 10 oleyl ether or polyoxyl 20 cetostearyl ether, polyethylene oxide, polyethylene glycol, peppermint oil, parraffin, olive oil, oleyl alcohol, oleic acid, octydodecanol, octoxynol 9, nonoxynol 10, myristyl alcohol, light mineral oil, lanolin alcohol, lanolin, isopropyl palmitate, isopropyl myristate, glyceryl monostearate, glyceryl behenate, oleate, cottonseed oil, cetyl alcohol, cetostearyl alcohol, cetyl esters wax, hydrogenated castor oil, butyl paraben, almond oil, soybean oil, simethicone, safflower oil, panthenol, mineral oil, common unsaturated fats including myristic, palmitic, stearic, oleic, linoleic, linolenic, arachidic, and eicosenoic acids and oils including cottonseed oil, canola oil, olive oil, corn oil, peanut oil, safflower oil, palm oil and sunflower oil.

3. A non-aqueous oral iodine composition as defined in claim 2, further comprising other iodine containing species wherein the ratio of total iodine to molecular iodine is in a range of between 0.9 and 1.0.

4. A non-aqueous oral iodine composition as defined in claim 3, wherein the ratio of total iodine to molecular iodine is between 0.95 and 1.0.
